(19) United States
(12) Patent Application Publication
(10) Pub. No.: US 2026/0315204 A1
(43) Pub. Date: Oct. 15, 2026

(54) CAPTURING FLOWING DEBRIS IN BLOOD VESSELS AND OTHER BODY LUMENS

(71) Applicant: Zeev BRANDEIS, Rosh HaAyin (IL)

(72) Inventor: Zeev BRANDEIS, Rosh HaAyin (IL)

(21) Appl. No.: 19/104,248

(22) PCT Filed: Aug. 17, 2023
(86) PCT No.: PCT/IL2023/050875

§ 371 Date: Feb. 17, 2025

Related U.S. Application Data
(60) Provisional application No. 63/398,546, filed on Aug. 17, 2022.
(60) Provisional application No. 63/400,866, filed on Aug. 25, 2022.

(51) Int. Cl. A61F 2/01; A61F 2/48

(52) U.S. Cl. CPC A61F 2/0105; A61F 2/48; A61F 2220/0008; A61F 2250/0014

(57) ABSTRACT

A device for capturing debris from blood flow in an aorta, the device including an expandable stent shaped and sized to expand against walls of a body lumen, a plurality of mesh debris traps attached to the stent, a control wire attached to one or more of the mesh debris traps, configured to control opening of the mesh debris traps. A method for collecting debris, the method including inserting a device for capturing debris from fluid flow to a body lumen, anchoring the device so as not to be moved downstream by the fluid flow, controlling mesh debris traps included in the device to open, and extracting the device from the body together with debris captured in the debris trap. Related apparatus and methods are also described.

### RELATED APPLICATIONS

This application is a PCT application which claims the benefit of priority of U.S. Provisional Patent Application No. 63/398,546 filed 17 Aug. 2022, and of U.S. Provisional Patent Application No. 63/400,866 filed 25 Aug. 2022, the contents of which are incorporated herein by reference in their entirety.

### FIELD AND BACKGROUND

The present disclosure, in some embodiments thereof, relates to methods and devices for trapping debris flowing along a lumen and, more particularly, but not exclusively, to methods and devices for trapping debris which are controlled to activate or deactivate the trapping from outside a body.

Blood borne debris such as emboli, calcification particles, plaque and detached plaque particles, tissue particles like myocardial tissue endothelial tissue and others, particles from foreign sources-metals, polymers and others can be dangerous, the debris may cause a pathological event, for example accompanying illness or injury or even medical procedures.

For example, in trans-catheter aortic valve implantation (TAVI), particles were found in 99% of patients by diffusion-weighted magnetic resonance imaging (DWI or DW-MRI). There was a significantly higher amount of debris related to the vascular bed (valve tissue, arterial wall, calcification), and additional particles: atherosclerotic plaque, myocardial tissue, endothelium tissue, and foreign particles of unknown source-such as polymers and air bubbles.

Additional background art includes:

International Patent Application Publication Number WO 2019/064223 of Brandeis, which describes an aortic protection device including a mesh lumen shaped and sized to extend along the aorta, from a heart-side of a brachiocephalic artery exit from the aorta to distal of a left subclavian artery exit from the aorta, wherein the mesh lumen is arranged to change a porosity of mesh pores in response to external control.

International Patent Application Publication Number WO 2017/042808 of Eli, which describes an embolic protection device including a porous deflector screen including a filter, arranged to expand and to conform to a wall of the aortic arch covering entrances to arteries branching from an aorta, an emboli collector including a cylinder arranged to expand and to lie along walls of a descending aorta, pushing against walls of the descending aorta and anchoring the porous deflector screen, and a connecting portion for connecting the porous deflector screen and the emboli collector, arranged to push the porous deflector screen against a wall of the aortic arch while anchoring against the emboli collector.

An article titled: “Debris Heterogeneity Across Different Valve Types Captured by a Cerebral Protection System During Transcatheter Aortic Valve Replacement—Focus On Stroke Risk And Prevention, by Tobias Schmidt et al., published in J. Am. Coll. Cardiol. Intv. 2018 July, 11 (13) 1262-1273.

The disclosures of all references mentioned above and throughout the present specification, as well as the disclosures of all references mentioned in those references, are hereby incorporated herein by reference.

### SUMMARY

The present disclosure, in some embodiments thereof, relates to methods and devices for trapping debris flowing along a lumen and, more particularly, but not exclusively, to methods and devices for trapping debris which are controlled to activate or deactivate the trapping from outside a body.

According to an aspect of some embodiments of the present disclosure there is provided a device for capturing debris from blood flow in an aorta, the device including an expandable stent shaped and sized to expand against walls of a body lumen, a plurality of mesh debris traps attached to the stent, a control wire attached to one or more of the mesh debris traps, configured to control opening of the mesh debris traps.

According to some embodiments of the disclosure, the expandable stent is shaped and sized to expand against walls of an aorta.

According to some embodiments of the disclosure, the expandable stent is shaped and sized to expand against walls of a Vena Cava.

According to some embodiments of the disclosure, the control wire is sized to extend from a location of the device in a patient's body to outside the patient's body.

According to some embodiments of the disclosure, the mesh debris traps are arranged to be controlled to open and close by the control wire from outside a patient's body.

According to some embodiments of the disclosure, at least some of the mesh debris traps are arranged to be controlled to open and close separately from at least some other mesh debris traps.

According to some embodiments of the disclosure, including a plurality of control wires, each control wire configured to control a separate group of the mesh debris traps.

According to some embodiments of the disclosure, the mesh debris traps are arranged to be normally closed even when the device is expanded against walls of the body lumen.

According to some embodiments of the disclosure, the mesh debris traps are configured to allow passage of medical tools through a tubular lumen defined by an inside of the device when the device is expanded against walls of the body lumen.

According to some embodiments of the disclosure, the mesh debris traps includes mesh leaves attached at their base to stent walls, and edges positioned upstream of the base of the mesh leaves.

According to some embodiments of the disclosure, the mesh debris traps includes mesh leaves attached at their base to stent walls, and edges positioned downstream of the base of the mesh leaves.

According to some embodiments of the disclosure, the mesh debris traps include markers to enable detecting whether the mesh debris traps are open or closed.

According to some embodiments of the disclosure, the markers include markers suitable for detection by imaging modalities, selected from a group consisting of x-ray, ultrasound, and magnetic resonance imaging (MRI).

According to some embodiments of the disclosure, the markers are located at mesh debris trap openings.

According to some embodiments of the disclosure, the mesh debris traps are arranged such that mesh debris trap openings are distributed at different distances along a direction of blood flow through the device.

According to some embodiments of the disclosure, at least some of the mesh debris trap levels are arranged to be closed separately from at least some other mesh debris trap levels.

According to some embodiments of the disclosure, the mesh debris traps include a mesh with pore sizes in a range between 1000 and 30 microns.

According to some embodiments of the disclosure, mesh debris trap openings distributed at different distances along a direction of blood flow include different sizes of mesh pore openings. According to some embodiments of the disclosure, mesh debris traps are arranged longitudinally along the device such that, when open, openings of open mesh debris traps overlap, as viewed along a direction of blood flow.

According to some embodiments of the disclosure, the different sizes of mesh pore openings are arranged such that larger pore sizes are upstream of smaller pore sizes.

According to some embodiments of the disclosure, including at least three levels of mesh pore size 1000 microns, 400 microns and 200 microns.

According to some embodiments of the disclosure, mesh debris trap openings at same distances along a direction of blood flow include different sizes of mesh pore openings.

According to some embodiments of the disclosure, when the plurality of mesh debris traps are opened, the trap openings cover an entire area of a cross section of a lumen defined by the expandable stent across a direction of blood flow.

According to some embodiments of the disclosure, mesh debris trap openings are shaped to conform to lumen walls when the mesh debris traps are not open.

According to some embodiments of the disclosure, mesh debris trap openings are shaped as arcs.

According to some embodiments of the disclosure, mesh debris trap openings are shaped as triangles.

According to some embodiments of the disclosure, mesh debris trap openings include a loop for threading a control wire therethrough, the control wire serving to open the mesh debris trap.

According to some embodiments of the disclosure, mesh debris trap openings are flexible, the mesh debris trap openings allowing surgical tools to bend them and pass along the device.

According to some embodiments of the disclosure, expansion of the stent against walls of the lumen anchors the device to resist movement along a direction of blood flow.

According to some embodiments of the disclosure, the device is anchored to resist movement along a direction of blood flow by connection to an anchor stent expanded against lumen walls upstream of the device.

According to some embodiments of the disclosure, the anchor stent is shaped and sized for anchoring upstream of a brachiocephalic trunk.

According to some embodiments of the disclosure, the anchor stent is shaped and sized for anchoring upstream of the carotid arteries.

According to some embodiments of the disclosure, the device is configured to attach to an aortic protection device.

According to some embodiments of the disclosure, the device is configured as a part of an aortic protection device.

According to an aspect of some embodiments of the present disclosure there is provided a method for collecting debris, the method including inserting a device for capturing debris from fluid flow to a body lumen, anchoring the device so as not to be moved downstream by the fluid flow, controlling mesh debris traps included in the device to open, and extracting the device from the body together with debris captured in the debris trap.

According to some embodiments of the disclosure, the anchoring includes anchoring downstream of an expected source of the debris.

According to some embodiments of the disclosure, the body lumen is a blood vessel.

According to some embodiments of the disclosure, the blood vessel is an artery.

According to some embodiments of the disclosure, the artery is the aorta.

According to some embodiments of the disclosure, further including inserting tools for performing a cardiac procedure along the device and upstream of the device.

According to some embodiments of the disclosure, further including performing the cardiac procedure following the opening of the mesh debris traps.

According to some embodiments of the disclosure, the device is used in addition to use of an aortic protection device.

According to some embodiments of the disclosure, the blood vessel is a vein.

According to some embodiments of the disclosure, the method is performed upon a patient who has a medical condition expected to release debris or produce debris into veins.

According to some embodiments of the disclosure, the method is performed upon a patient prior to performing a medical procedure expected to release debris or produce debris into veins.

According to some embodiments of the disclosure, further including releasing the anchoring of the device, re-positioning the device, and re-anchoring the device.

According to some embodiments of the disclosure, further including closing the mesh debris traps prior to extracting the device from the body.

According to some embodiments of the disclosure, the closing the mesh debris traps is done following the performing of the cardiac procedure.

According to some embodiments of the disclosure, the opening the mesh debris traps includes controlling just a sub-group of the mesh debris traps.

According to an aspect of some embodiments of the present disclosure there is provided a method for collecting debris during a cardiac procedure, the method including inserting a device for capturing debris to a location in the aorta, inserting a medical tool for performing the cardiac procedure, performing the cardiac procedure, and extracting the device for capturing debris from the body together with debris captured in debris traps in the device.

According to some embodiments of the disclosure, the device debris traps in the device are controlled to close prior to the extracting.

According to some embodiments of the disclosure, the debris traps in the device are controlled to open prior to performing the cardiac procedure.

According to some embodiments of the disclosure, the cardiac procedure is a cardiac procedure selected from a group consisting of electrophysiology procedures, Patent Foramen Ovale (PFO) procedures, heart valve repairs, open heart surgery, percutaneous aortic valve replacement (PAVR), percutaneous aortic valve implantation (PAVI), transcatheter aortic valve implantation (TAVI), and transcatheter aortic valve replacement (TAVR).

According to some embodiments of the disclosure, the debris traps in the device are controlled to open following or during a procedure selected from a group consisting of an aneurism procedure, an atherosclerosis stenting procedure, a balloon dilation procedure, a drug delivery procedure, a kidney procedure, a surgery procedure involving treating an artery, and an atheromatous aorta treatment.

According to some embodiments of the disclosure, the cardiac procedure includes a pace-up step, and the cardiac procedure debris traps in the device are controlled to open after the pace-up step.

According to some embodiments of the disclosure, the cardiac procedure includes a pace-down step, and the cardiac procedure debris traps in the device are controlled to open after the pace-down step.

According to an aspect of some embodiments of the present disclosure there is provided a method for preventing debris from reaching a lung, the method including inserting a device for capturing debris to a location in a vein, controlling debris traps in the device to open and collect debris from the vein, performing the medical procedure, and extracting the device for capturing debris from the body.

According to some embodiments of the disclosure, prior to extracting the device the debris traps are controlled to close.

According to some embodiments of the disclosure, the extracting includes extracting together with debris captured in debris traps in the device.

According to some embodiments of the disclosure, further including administering an anti-coagulant before the extracting.

According to some embodiments of the disclosure, the debris traps in the device are controlled to open following or during a procedure selected from a group consisting of a kidney procedure, an aneurism procedure, an injury, an open injury, an amputation, injuries caused in a disaster setting, an injury caused by trauma, an injury caused by blunt trauma, a medical procedure which includes heart assist, a medical procedure which includes lung assist, a medical procedure which includes ExtraCorporeal Membrane Oxygenation (ECMO), an open surgery, and an injury caused by pressure.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the disclosure are herein described, by way of example only, with reference to the accompanying drawings and/or images. With specific reference now to the drawings and/or images in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified block diagram illustration of debris flowing downstream and a device placed to trap the debris, according to an example embodiment;

FIG. 1B is a simplified block diagram illustration of debris flowing downstream and a device placed to trap the debris, according to an example embodiment;

FIGS. 2A-2F are simplified schematic line drawing illustrations of debris capture devices according to some example embodiments;

FIG. 3A is a simplified schematic line drawing illustrations of a debris capture device according to an example embodiment;

FIG. 3B is a simplified schematic line drawing illustration of a debris capture device according to an example embodiment;

FIGS. 4A-4C are simplified line drawing illustrations of an example embodiment of a debris capture device;

FIG. 5A is a photo of a debris capture device according to an example embodiment;

FIG. 5B is a simplified illustration of a debris capture device according to an example embodiment;

FIGS. 6A-6C are photos of a debris capture device according to an example embodiment;

FIGS. 7A-7C are simplified illustrations of a debris capture device located in an aorta according to an example embodiment;

FIG. 7D is a simplified illustration of a debris capture device located in an aorta according to an example embodiment;

FIG. 7E is a simplified illustration of a debris capture device located in an aorta according to an example embodiment;

FIG. 7F is a simplified line drawing illustration of a device for aortic protection deployed in conjunction with a debris trapping device according to an example embodiment of the invention; FIGS. 7G and 7H are simplified line drawing illustrations of a device which includes a combination of aortic protection and debris trapping according to an example embodiment of the invention;

FIGS. 7I and 7J are simplified line drawing illustrations of a device which includes a combination of aortic protection and two locations or levels for debris trapping according to an example embodiment of the invention;

FIG. 8 is a simplified illustration of potential locations for a debris trapping device in an aorta according to some example embodiments;

FIG. 9 is a simplified illustration of a potential location for a debris trapping device in a vein according to some example embodiments;

FIG. 10 is a simplified flow chart illustration of a method for collecting debris according to an example embodiment;

FIG. 11 is a simplified flow chart illustration of a method for collecting debris during a cardiac procedure according to an example embodiment; and

FIG. 12 is a simplified flow chart illustration of a method for preventing debris from reaching a lung according to an example embodiment.

### DESCRIPTION OF SPECIFIC EMBODIMENTS

The present disclosure, in some embodiments thereof, relates to methods and devices for trapping debris flowing along a lumen and, more particularly, but not exclusively, to methods and devices for trapping debris which are controlled to activate or deactivate the trapping from outside a body.

### Overview—Embodiments of a Device

An aspect of some embodiments relates to a device for trapping debris flowing through a lumen in a body, for example debris flowing through a blood vessel.

The term debris is used throughout the present specification and claims to include blood clots, emboli, micro-emboli, Calcification particles, body tissue particles, myocardial tissue particles, polymer particles, polymer micro-particles, foreign particles in body lumens, endothelium tissue particles, unorganized blood clots, organized blood clots, plaque particles, particles flowing in the blood stream and other undesirable particles found in body fluids. The term debris also includes stones from various body sources-kidney stones, gallbladder stones, spleen, pancreatic stones and others.

In some embodiments, the device has a control which can activate or deactivate the trapping, that is, activate debris trapping pockets to a state where the pockets trap debris, and deactivate the debris trapping pockets to close to a state where the pockets close. In some embodiments, the debris trapping pockets include a mesh, optionally a flexible mesh, with a specific pore size, intended to trap debris larger than the specific pore size.

The term “mesh” is used throughout the present specification and claims to mean material having pores or holes which let particles smaller than the pores/holed pass therethrough.

The term “mesh” is used throughout the present specification and claims interchangeably with the terms membrane, film, knitted fabric, polymer, electro-spun polymer, nitinol mesh, and bio-degradable membranes of polymer and/or biological origin.

In some embodiments, the control extends from the device to outside a patient's body. In some embodiments, the control is a wire, which, when acted upon outside the body, causes the debris trapping pockets to open and/or close.

In some embodiments, when the pockets are closed, the pockets close on any debris within the pockets, keeping the debris within the pockets, even over a procedure of withdrawing the device from the body. Such an embodiment enables extracting the debris from the body.

In some embodiments, the device has debris trapping pockets arranged around an inside circumference of a stent. When the pockets are activated, that is, opened, the pockets optionally extend toward a center of the stent, optionally completely covering a cross-section of the stent. In some embodiments, when the pockets extend toward the center of the stent, the pockets are flexible enough to lie against a medical tool which extends through the stent, optionally sealing a cross section of the stent against the medical tool. In some embodiments, when the pockets extend toward the center of the stent, the pockets are flexible enough to lie as a curtain against a medical tool which extends through the stent, optionally sealing a cross section of the stent against the medical tool.

In some embodiments, the debris trapping pockets are normally closed, that is, fluid can flow past the debris trapping pockets with low resistance, and the fluid is not filtered through the debris trapping pockets. A state where the debris trapping pockets are closed is also called a state where the device for capturing debris is open (is not activated for trapping debris). A potential advantage of such an embodiment can be that fluid flow is unhampered until there is a cause to filter and/or trap debris. In some embodiments, the trapping may be activated only during a medical procedure which is known as generating debris. One non-limiting example of such use can be when the device is placed in an aorta, and activated during performance of a cardiac procedure, or during a pace-down following the cardiac procedure, when blood flow is known to increase, potentially increasing debris flow. It is potentially advantageous to allow blood to flow unimpeded until a time which is known or suspected to generate an increase in debris flowing downstream.

In some embodiments, the debris trapping pockets are normally open, that is, fluid is filtered through the debris trapping pockets. A state where the debris trapping pockets are open is a state where the device for capturing debris is activated for trapping debris. A potential advantage of such an embodiment can be that fluid is constantly filtered to trap debris, there is no need to decide when to start trapping. In some embodiments, the trapping may last along all or most of the duration that the device is deployed in place. One non-limiting example of such use can be when the device is placed in a vein, to prevent debris from reaching a patient's lungs. In some embodiments, the device is optionally placed in a vein of a patient who has suffered from an accident which crushed limbs. Such an accident is known to produce blood clots and/or other debris which mat flow to the lungs, and using the device in a vein such as the Vena Cava may trap debris and prevent the debris from reaching the lungs. In some embodiments, such prevention can last all along a medical procedure performed on a patient when there is concern that the medical procedure produces debris.

In some embodiments, the debris trapping pockets are activated to open and filter and/or trap debris out of fluid flow. In some embodiments, the activation requires a physician to continually exert force to hold the debris trapping pockets open, and when the physician ceases to exert force, the debris trapping pockets close.

In some embodiments, the pockets are arranged at several distances along an axial direction of the stent. When the pockets are activated, the pockets optionally extend toward a center of the stent, optionally completely covering a cross-section of the stent at each one of the several distances, also called several levels, along the axial direction of the stent. A potential benefit of constructing the device with several levels of pockets can be that the trapped debris is distributed along the stent, and potentially, when the device is extracted from a body, carrying the trapped debris with it in closed pockets, the debris is distributed, so that the device comes out longer and thinner than if all the trapped debris was at one level.

In some embodiments, the control is configured to control all the levels of the pockets. In some embodiments, the control is configured to control one or more of the levels of the pockets separately from other levels of the pockets. In some embodiments, the control includes a separate control for each level of the pockets.

In some embodiments, each one of the several levels includes pockets made of mesh with a different pore size. In some embodiments, the several levels are arranged so that larger sized pores are configured to be placed upstream, relative to flow direction in the stent, than smaller sized pores. Such embodiments potentially provide a benefit of trapping different sized debris at different distances along the stent, potentially distributing the trapping of different sized debris along the different levels, and potentially, when the device is extracted from a body, carrying the trapped debris with it in closed pockets, the debris is distributed, so that the device comes out longer and thinner than if all the trapped debris was at one level.

### Overview—Potential Locations of the Device

An aspect of some embodiments relates to a location in a lumen of a body in which a device for trapping debris flowing through the lumen is located.

Typically, the device includes a stent which can expand to a diameter of the lumen, so as to push against the lumen walls. In some embodiments, the expansion seals the stent against the lumen walls, not allowing debris to flow between the stent and the lumen walls. In some embodiments, the expansion anchors the stent in place, preventing the stent from sliding along the lumen walls, even when the debris trapping pockets are engaged, and fluid flow along the lumen potentially exerts pressure on the pockets to push to stent downstream.

Some non-limiting example locations are now described with reference to trapping debris in a blood stream, in which case the lumens through which the blood flows are arteries and/or veins. An example location may be the descending aorta. The descending aorta is an artery which is relatively large in diameter, and provides a relatively long section of lumen in which to place an embodiment of the device.

An example location may be the abdominal aorta.

As described above, embodiments of the device may include one or more levels of debris trapping pockets, and may thus be shorter or longer, potentially suitable for different locations. For example, when placing in the descending aorta, the device embodiment may include more than one level of pockets.

An example location may be the aortic arch. The descending aorta is an artery which is relatively large in diameter, and provides a curved section of lumen in which to place an embodiment of the device.

An example location may be the ascending aorta, for example between the cardiac arteries and the brachiocephalic trunk.

An example location may be the Vena Cava, before blood enters the heart and is pumped to the lungs, where debris may cause serious harm.

### Overview—Non-limiting Example Method of Use

An aspect of some embodiments relates to a method of using the debris trapping device.

In some embodiments, the device is inserted into a body, and advanced along a body lumen to an intended location. When the device reaches the location, a stent included in the device is expanded, to push against the lumen walls. By way of a non-limiting example, the stent may include memory material, so that when the stent is pushed out of a catheter, the stent expands.

In some embodiments, a catheter is used to insert the device into the body. In some embodiments, the catheter may optionally be a standard vascular access catheter. Some contemplated catheter diameters include diameters in a range from 5 French to 12 French. In some embodiments, the catheter may optionally be a small bore femoral access catheter. Some contemplated catheter diameters include diameters in a range from 5 French to 8 French.

In some embodiments, the device is designed for Radial access.

In some embodiments, the debris trapping device is optionally inserted from a same femoral access as tools for an additional medical procedure (such as a balloon-stent graft etc.) or from a patient's other leg, that is, a femoral access that is not via a same leg as access for the additional medical procedure.

Optionally, in some embodiments, a medical tool may be passed through and/or along the debris trapping device, to perform a medical procedure upstream of the debris trapping device.

At a suitable time, when there is a need to trap debris, the debris trapping pockets are optionally activated. In some embodiments, the pockets extend toward the center of the stent, covering the cross section of the lumen, so that all fluid flowing along the lumen passes through the debris trapping pockets. In some embodiments, the pockets extend toward the center of the stent, lying along a catheter used to lead medical tool(s) upstream through the device, closing off the cross section of the lumen, so that fluid flowing along the lumen passes through the debris trapping pockets.

A typical time when there is a need to trap debris is during a surgical or other medical operation which may release debris particle.

By way of a non-limiting example, a debris trapping device is optionally activated when an operation is performed on a heart, by way of a non-limiting example on a mitral valve, such an operation may release particles of sediment which may have accumulated on the mitral valve, such particles are optionally trapped, instead of being allowed to flow along arteries, which might reach and might harm kidneys, the brain, the heart.

By way of a non-limiting example, a debris trapping device is optionally activated when an operation is performed on a body. Such an operation may release emboli, such as blood clots, such particles are optionally trapped before entering the right side of the heart, instead of allowing the heart to pump the particles toward the lungs, which might harm the lungs.

### Overview—Use in Relation to a Medical Procedure

An aspect of some embodiments includes using a debris trapping device downstream of a location of performing a medical procedures. In some embodiments, a medical procedure may release debris to flow with a body fluid, such as, by way of a non-limiting example, blood, and the debris trapping device is optionally activated before starting to perform the medical procedure, so as to trap the debris and prevent the debris from flowing and arriving at locations in a body where the debris may cause harm.

In some embodiments, when a medical procedure is performed upon a heart, an embodiment of the debris trapping device is optionally deployed downstream of the heart, for example in the aorta, so as to trap debris caused by the procedure.

By way of a non-limiting example, a debris trapping device is optionally activated when an operation is performed on a mitral valve, such an operation typically releases particles of sediment which may have accumulated on the mitral valve, such particles are optionally trapped, instead of being allowed to flow along arteries, which might reach and might harm kidneys, the brain, the heart.

In some embodiments, when a medical procedure is performed on a body, debris may be released and flow along veins toward the heart. The debris may include emboli, for example blood clots. If the debris is allowed to enter from the venous system into the heart, the heart will pump the debris toward the lungs. Emboli in the lungs may cause damage. In some embodiments, a debris trapping device is placed at an entrance to the heart, for example in the Vena Cava, to prevent debris from entering the heart and continuing on toward the lungs. In some embodiments, the debris trapping device in the vein is optionally activated before starting a medical operation.

In some embodiments, when a body has been involved in an accident, blood clots may build up in a body part which has been involved in the accident. A debris trapping device may optionally be placed at an entrance to the heart, for example in the Vena Cava, to prevent debris from entering the heart and continuing on toward the lungs. In some embodiments, the debris trapping device in the vein is optionally activated before starting a medical operation intended to treat the body part damaged in the accident.

### Overview—Relation to Other Devices

An aspect of some embodiments relates to a debris trapping device attached to and/or constructed together with, additional medical devices.

By way of a non-limiting example, an embodiment of the debris trapping device may optionally be attached to and/or part of an aortic protection device as described in above-mentioned International Patent Application Publication Number WO 2019/064223 of Brandeis.

An aspect of some embodiments relates to a debris trapping device used in addition to use of additional medical devices.

By way of a non-limiting example, an embodiment of the debris trapping device may optionally be used in addition to use of an aortic protection device as described in above-mentioned International Patent Application Publication Number WO 2019/064223 of Brandeis.

By way of a non-limiting example, an embodiment of the debris trapping device may optionally be used in addition to tools used for percutaneous aortic valve replacement (PAVR), also known as percutaneous aortic valve implantation (PAVI), transcatheter aortic valve implantation (TAVI) or transcatheter aortic valve replacement (TAVR).

In some embodiments, the debris trapping device is optionally deployed first, and the above-mentioned tools optionally pass through the debris trapping device. In some embodiments, the above-mentioned tools optionally pass through the debris trapping device even while the debris trapping pockets are activated. In some embodiments, lips or edges of the pockets are flexible enough to allow the tools through, optionally maintaining contact with the tools and filtering blood and potentially trapping debris.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

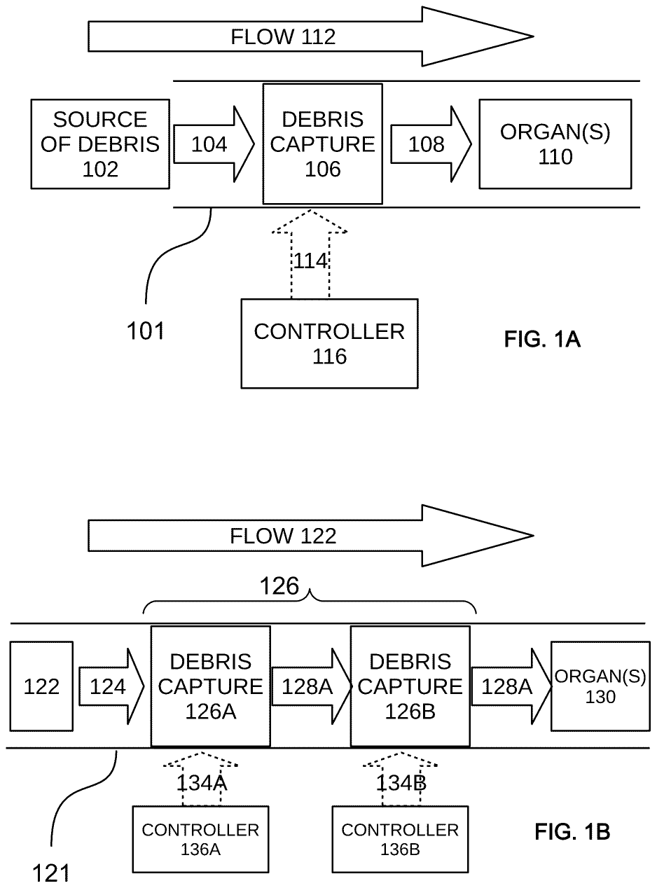

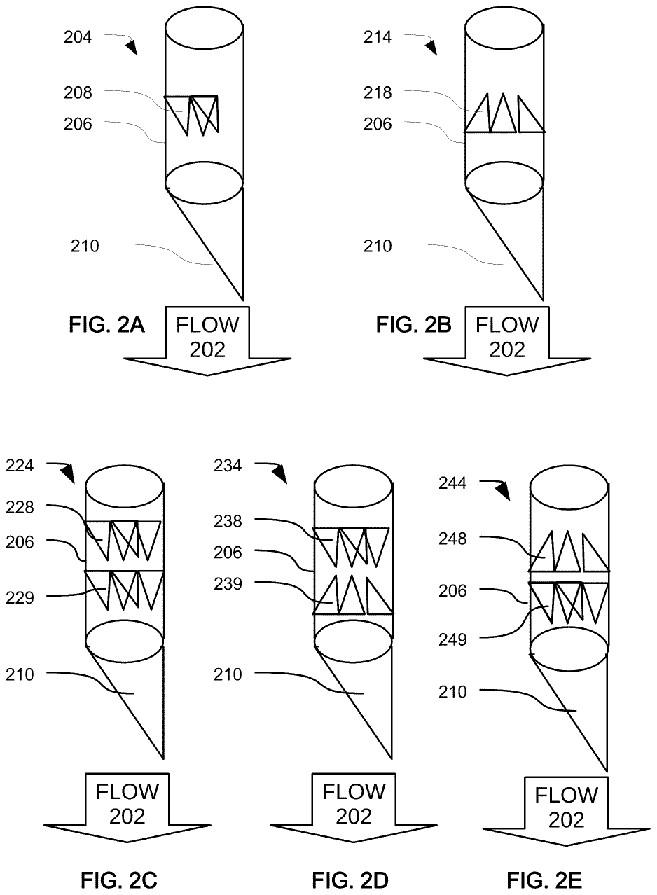

Reference is now made to FIG. 1A, which is a simplified block diagram illustration of debris flowing downstream and a device placed to trap the debris, according to an example embodiment.

FIG. 1A shows some source of debris 102 (for example calcified heart valve leaflets releasing calcium flakes during a medical operation, or blood emboli from a crushed tissue) releasing debris which flows 104 along a body lumen 101 (for example an artery such as an aorta, or a vein such as the Vena Cava). The debris reaches a debris capture device 106. Downstream of the debris capture device 106 fluid (such as blood) continues to flow 108, less some or all of the debris, which has been trapped by the debris capture device 106. The fluid reaches organs 110, which potentially benefit from the debris not reaching them. Such organs may be, by way of some non-limiting examples, the lungs (in which case the debris capture device 106 was placed in a vein, optionally in the Vena Cava), may be the heart, and/or the brain (in which case the debris capture device 106 was placed in the aorta), and/or the kidneys (in which case the debris capture device 106 was placed in the aorta or the abdominal aorta).

In some embodiments, the debris capture device 106 optionally has at least two states-an open state which does not trap debris, potentially allowing fluid through with maximal flow, minimal resistance to flow, and a closed state, which captures debris, potentially allowing a cleaner fluid with less or nor debris thein to flow through and reach the organs 110.

In some embodiments, the debris capture device 106 includes debris trapping leaves or pockets which extend across a cross section of the debris capture device 106, where the trapping leaves or pockets include a mesh material which filters fluid through, and blocks debris from passing through.

The mesh material may include a mesh with a pore size of 1000, 200, 150, 130, 100, 75, 50, 30 microns, down to 10 microns.

In some embodiments, the debris capture device 106 is optionally controlled 114 by a controller 116 to switch between the two states.

In some embodiments, the controller 116 is optionally activated by a physician.

In some embodiments, the controller 116 is optionally automatically activated by functionally connecting to a sensor and programming activation of the debris capture device 106 based on the sensor reading.

By way of one non-limiting example, during a TAVI operation, the heart pace is artificially raised (called pace-up), which causes the heart to pump less blood, at which point the aortic valve implantation begins. The process of implantation may release debris into the blood flow. At such a time (pace-up) it may be desired that the debris capture device 106 be automatically activated to capture debris, preventing flow of the debris downstream to organs which might potentially suffer from debris in the blood.

By way of one non-limiting example, during a TAVI operation, following implantation, the heart pace is lowered back (called pace-down), which causes the heart to pump more blood. The increased blood flow may cause debris to flow from the site of the medical procedure. At such a time (pace-down) it may be desired that the debris capture device 106 be automatically activated to capture debris, preventing flow of the debris downstream to organs which might potentially suffer from debris in the blood.

A sensor suitable to participate in automatically activating the debris capture device 106 can be a sensor which provides a heart pace reading.

In some embodiments, the heart pace sensor may optionally be located separate from the debris capture device 106, and provide data to the controller 116.

In some embodiments, a physician manually activates and/or deactivates the controller 116 whether according to the rationale described above with reference to automatic activation, or to a rationale associated with a method for a specific medical operation.

In some embodiments, a physician manually activates and/or deactivates the controller 116 based on specific activities related to a medical procedure being performed. By way of one non-limiting example, when medical tools are being moved along a lumen, for example along the aorta, the movement may release particles, and the debris trapping device may optionally be activated to trap the particles. By way of another non-limiting example, when medical tools are being repositioned, the movement may release particles, and the debris trapping device may optionally be activated to trap the particles. By way of yet another non-limiting example, when a balloon is planned to be inflated/expanded, the expansion may release particles, and the debris trapping device may optionally be activated to trap the particles.

In some embodiments, the controller 116 may optionally be a control wire 116. In some embodiments, the control wire 116 is optionally attached at one end to the debris capture device 106, and another end extend to outside a patient's body.

Reference is now made to FIG. 1B, which is a simplified block diagram illustration of debris flowing downstream and a device placed to trap the debris, according to an example embodiment.

FIG. 1B shows some source of debris 122 releasing debris which flows 124 along a body lumen 121. The debris reaches a debris capture device 126 which includes several debris capture traps 126A 126B (two or more) or groups of traps 126A 126B. Downstream of a first debris capture trap 126A fluid continues to flow 128A, less some or all of the debris, which has been trapped by the debris capture trap 126A.

In some embodiments, the fluid 128A reaches a second debris capture trap 126B following which the fluid continues to flow 128B, less some or all of the debris, which has been trapped by the debris capture trap 126B.

The fluid reaches organs 130, which potentially benefit from the debris not reaching them.

In some embodiments, the debris capture device 126 is optionally controlled 134A 134B by one or more controllers 136A 136B to switch between OPEN and CLOSED states.

In some embodiments, the debris capture device 126 is optionally controlled by just one controller.

In some embodiments, one or more of the debris capture traps 126A 126B or groups of traps 126A 126B are optionally controlled separately. In some embodiments, the debris capture traps 126A 126B or groups of traps 126A 126B are controlled independently of each other.

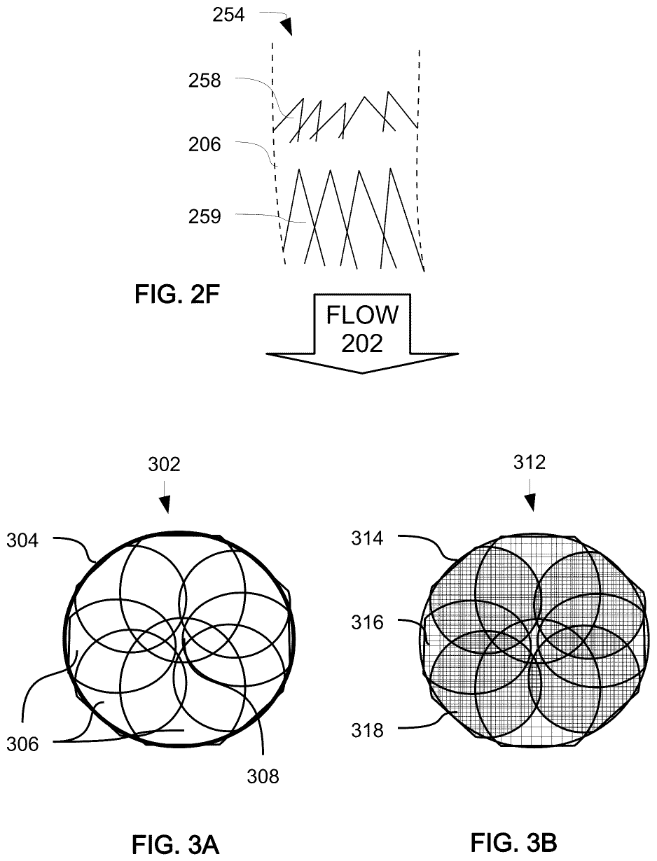

Reference is now made to FIGS. 2A-2F, which are simplified schematic line drawing illustrations of debris capture devices according to some example embodiments.

FIG. 2A shows a debris capture device 204 including a stent 206, having leaves 208 or debris traps 208, or debris catching pockets 208, which can open (lie against walls of the stent 206, and close (bend toward a center of a lumen defined by the stent 206). FIG. 2A also shows an optional wire 210 or wires 210 which in some embodiments, serve to control activation the debris catching pockets 208. FIG. 2A also shows a direction of fluid flow 202.

FIG. 2A shows a debris capture device 204 with bases of the leaves 208 attached to the stent 206, and free edges of the leaves 208 pointing upstream in relation to the direction of fluid flow 202.

FIG. 2B shows a debris capture device 214 including a stent 206, having leaves 218 or debris traps 218, or debris catching pockets 218, which can open (lie against walls of the stent 206, and close (bend toward a center of a lumen defined by the stent 206). FIG. 2B also shows an optional wire 210 or wires 210 which in some embodiments, serve to control activation the debris catching pockets 218. FIG. 2B also shows a direction of fluid flow 202.

FIG. 2B shows a debris capture device 214 with bases of the leaves 218 attached to the stent 206, and free edges of the leaves 218 pointing downstream in relation to the direction of fluid flow 202.

FIG. 2C shows a debris capture device 224 including a stent 206, having leaves or debris traps, or debris catching pockets 228 229 at two different levels relative to a direction of fluid flow 202 along a lumen defined by the stent 206. The leaves or debris traps, or debris catching pockets 228 229 can open (lie against walls of the stent 206, and close (bend toward a center of a lumen defined by the stent 206). FIG. 2C also shows an optional wire 210 or wires 210 which in some embodiments, serve to control activation the debris catching pockets 228 229.

FIG. 2C shows the debris capture device 224 with bases of the leaves 228 attached to the stent 206, and free edges of the leaves 228 pointing upstream in relation to the direction of fluid flow 202.

FIG. 2D shows a debris capture device 234 including a stent 206, having leaves or debris traps, or debris catching pockets 238 239 at two different levels relative to a direction of fluid flow 202 along a lumen defined by the stent 206. The leaves or debris traps, or debris catching pockets 238 239 can open (lie against walls of the stent 206, and close (bend toward a center of a lumen defined by the stent 206). FIG. 2D also shows an optional wire 210 or wires 210 which in some embodiments, serve to control activation the debris catching pockets 238 239.

FIG. 2D shows the debris capture device 234 with bases of the leaves 238 attached to the stent 206, and free edges of the leaves 238 pointing upstream in relation to the direction of fluid flow 202, and bases of the leaves 239 attached to the stent 206, and free edges of the leaves 239 pointing downstream in relation to the direction of fluid flow 202. The two levels of the leaves 238 239 point in different directions, the free edges pointing toward each other.

FIG. 2E shows a debris capture device 244 including a stent 206, having leaves or debris traps, or debris catching pockets 248 249 at two different levels relative to a direction of fluid flow 202 along a lumen defined by the stent 206. The leaves or debris traps, or debris catching pockets 248 249 can open (lie against walls of the stent 206, and close (bend toward a center of a lumen defined by the stent 206). FIG. 2D also shows an optional wire 210 or wires 210 which in some embodiments, serve to control activation the debris catching pockets 248 249.

FIG. 2D shows the debris capture device 244 with bases of the leaves 248 attached to the stent 206, and free edges of the leaves 248 pointing downstream in relation to the direction of fluid flow 202, and bases of the leaves 249 attached to the stent 206, and free edges of the leaves 249 pointing upstream in relation to the direction of fluid flow 202. The two levels of the leaves 248 249 point in different directions, the free edges pointing away from each other.

FIG. 2F shows a debris capture device 254 including a stent 206, having leaves or debris traps, or debris catching pockets 258 259 at two different levels relative to a direction of fluid flow 202 along a lumen defined by the stent 206. The leaves or debris traps, or debris catching pockets 258 259 can open (lie against walls of the stent 206, and close (bend toward a center of a lumen defined by the stent 206).

FIG. 2F shows the debris capture device 254 with bases of the leaves 258 259 attached to the stent 206, and free edges of the leaves 258 259 pointing downstream in relation to the direction of fluid flow 202.

FIG. 2F is also intended to how that a length of the leaves 258 at one level may not be equal to a length of the leaves 259 at another level.

It is noted that more than two levels of the leaves, as shown in FIGS. 2C-2E, may be used.

In some embodiments, the different levels of debris trapping leaves or pockets may have different pore sizes of a mesh with which the leaves filter fluid and capture debris.

In some embodiments, the different pore sizes of the different levels are arranged so that the larger pore sizes are upstream of the smaller pore sizes.

Potentially, by trapping different sizes of debris at different levels, one or more potential benefits may be realized:

Different sizes of debris are captured at different levels. When and if the debris traps are closed, and if the device is optionally withdrawn from a patient's body, the debris may be distributed according to debris size along the different levels, so the withdrawing is of a longer and narrower device, which may potentially injure the patient less.

Trapping smaller sizes of debris, by filtering through smaller pore sizes, potentially impedes fluid flow, for example blood flow-which may have a negative effect on a patient. In some embodiments, the smaller pore sizes may be used for a short time, possible when a maximum flood of debris is expected, and the larger pore sizes may be used for a longer time, potentially preventing bigger debris particles from flowing downstream.

In some embodiments, the different pore sizes of different traps are controlled separately so that the larger pore size traps are controlled separately from the smaller pore size traps.

In some embodiments, the different pore sizes of different levels are controlled separately so that the larger pore sizes are controlled separately from the smaller pore sizes.

In some embodiments, a physician selects which pore size traps to activate at which time during a medical procedure.

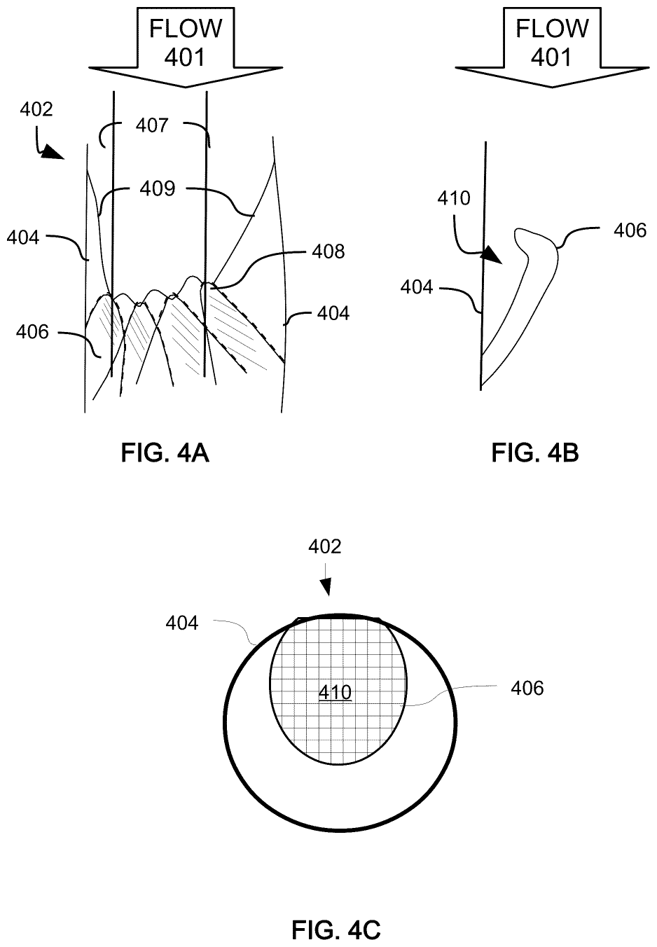

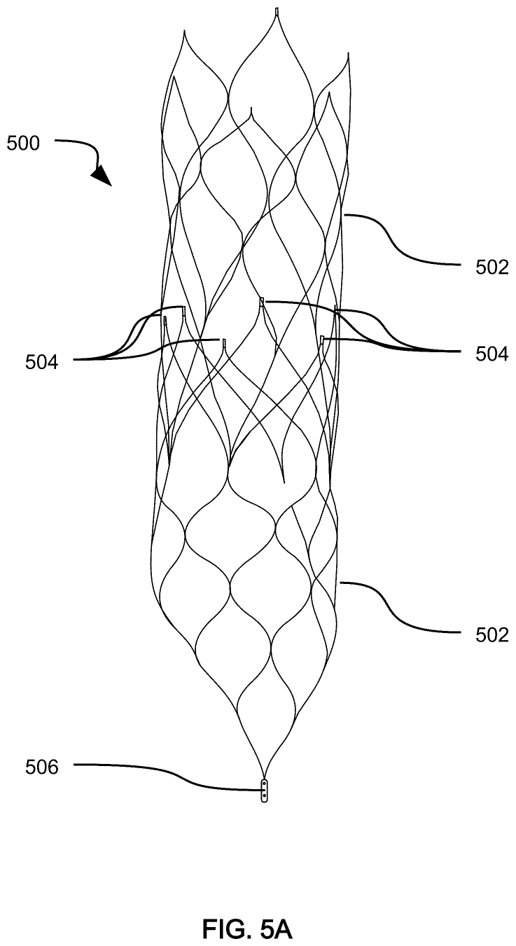

Reference is now made to FIG. 3A, which is a simplified schematic line drawing illustrations of a debris capture device according to an example embodiment.

FIG. 3A shows a debris capture device 302, including a stent 304 and debris capture leaves 306 open, in an activated state.

FIG. 3A is a view along a lumen defined by the stent 304, showing a cross section of the debris capture device 302 parallel to a direction of fluid flow, either in the direction of fluid flow, or opposite the direction of fluid flow.

FIG. 3A shows that in some embodiments, when the debris capture leaves 306 are open, an entire cross section area of the debris capture device 302 is covered.

In some embodiments, the debris capture leaves 306 are covered by a mesh having a pore size selected according to size of debris which should not pass through the debris capture device 302.

FIG. 3A also shows that, if needed, medical tools can pass through a center 308 of the debris capture device 302 by pushing aside the debris capture leaves 306.

In some embodiments, the debris capture leaves 306 are flexible, and lie against the medical tools and continue to filter fluid, filtering the cross section area around the medical tools.

Reference is now made to FIG. 3B, which is a simplified schematic line drawing illustration of a debris capture device according to an example embodiment.

FIG. 3B shows a debris capture device 312, including a stent 314 and debris capture leaves 316 318 open, in an activated state.

FIG. 3B is a view along a lumen defined by the stent 314, showing a cross section of the debris capture device 312 parallel to a direction of fluid flow, either in the direction of fluid flow, or opposite the direction of fluid flow.

FIG. 3B shows that in some embodiments, when the debris capture leaves 316 318 are open, an entire cross section area of the debris capture device 312 is covered.

In some embodiments, the debris capture leaves 316 are covered by a mesh having a first pore size, and the debris capture leaves 318 are covered by a mesh having a second, different, pore size.

In some embodiments, it is enough that only one group of debris capture leaves are open, for examples only the debris capture leaves 316, or only the debris capture leaves 318, to cover the entire cross section area of the debris capture device 312.

In some embodiments, the debris capture leaves having different pore-size meshes are optionally separately controllable. The debris capture device 312 may be activated with just the first pore size, or with just the second pore size, or with both pore sizes.

By way of a non-limiting example, a pore size of 200 microns may optionally be used to protect locations downstream of the debris capture device.

By way of a non-limiting example, a pore size of 100 microns, or less, for example 30 microns, may optionally be used to protect locations downstream of the debris capture device in cases with specific medical indications such as protecting kidneys in case of a patient whose kidneys are especially in danger, protecting the liver in case of a patient whose liver is especially in danger, protecting pelvic organs in case of a patient whose pelvic organs are especially in danger, protecting the lungs by trapping debris from entering the right ventricle of the heart and from there the lungs, in case of medical operations on limbs, pelvis, and so on.

FIG. 3B also shows that, if needed, medical tools can pass through a center of the debris capture device 312 by pushing aside the debris capture leaves 316 318.

In some embodiments, one or more of the debris capture leaves 316 318 is optionally controlled to change a mesh pore size of the debris capture leaves 316 318. By way of a non-limiting example, the debris capture leaves 316 318 may optionally be elongated, for example by pulling on a free edge or tip of the debris capture leaves 316 318. When the free edge or tip of the debris capture leaves 316 318 is pulled and the leaves are elongated, the pores are reshaped, and smaller debris particles are optionally trapped by the debris capture leaves 316 318.

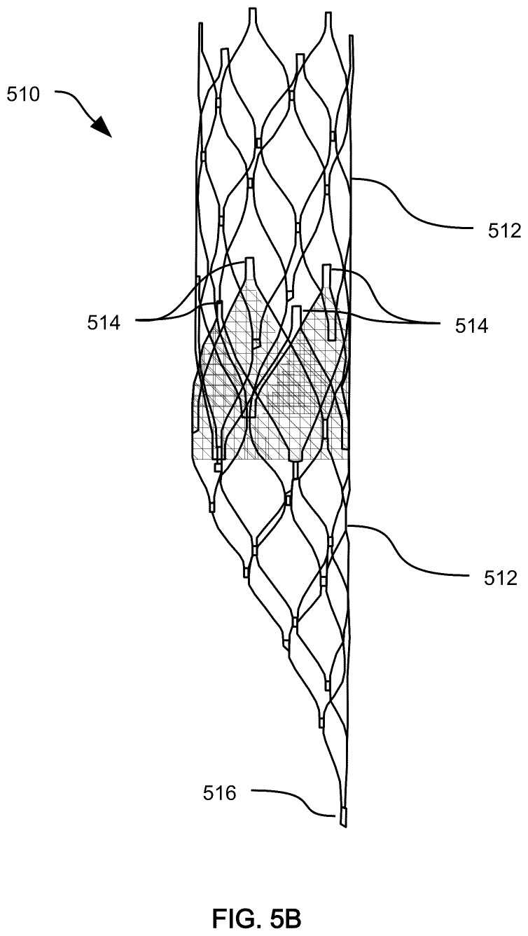

Reference is now made to FIGS. 4A-4C, which are simplified line drawing illustrations of an example embodiment of a debris capture device.

FIG. 4A shows a debris capture device 402 including a stent 404, having leaves 406 or debris traps 406, or debris catching pockets 406.

FIG. 4A shows tips 408 of the leaves 406, optionally lying against a medical tool 407 which passes through the debris capture device 402.

FIG. 4A also shows an example direction of fluid flow 401.

In some embodiments, the debris catching pockets 406 optionally include markers to enable detecting whether the debris traps are open or closed. In some embodiments, markers are markers suitable for detection by various imaging modalities, such as x-ray and/or ultrasound and/or magnetic resonance imaging (MRI). In some embodiments, the markers are optionally placed at the tips 408.

FIG. 4A shows an embodiment of debris traps with a centermost tip 408 of the trap and a debris trap or debris pocket lip 409 extending toward the stent 404 wall.

FIG. 4B shows a portion of the debris capture device 402, the portion including one wall of the stent 404, and one of the leaves 406. FIG. 4B shows a pocket 410 or debris trap 410, into which debris can flow along with the fluid flow 401. The leaf 406 filter the fluid through, and traps particles of debris which are larger than a pore size of mesh on the leaf, in a pocket-shaped trap 410.

When the debris capture device 402 is optionally deactivated, the traps close, containing trapped debris within the traps.

In some embodiments, the debris capture device 402 is optionally withdrawn from a patient's body, together with the trapped debris.

FIG. 4C shows a cross section of the stent 404, and one debris capture trap 410 of the debris capture device 402. The debris capture trap 410 is shown open, extending toward a center of the stent 404.

In some embodiments, the debris capture trap 410 may extend beyond the center of the stent 404, yet be flexible enough to allow medical tools to push the debris capture trap 410 slightly aside, to pass through.

In some embodiments, when several debris capture traps 410 are opened, the debris capture traps 410 optionally cover the entire cross section of the stent 404, and still allow medical tools to push the debris capture traps 410 slightly aside, to pass through.

In some embodiments, the debris capture traps 410 may extend up to the center of the stent 404, or less than the center of the stent 404, and when several debris capture traps 410 are opened, the debris capture traps 410 optionally cover the entire cross section of the stent 404, and still allow medical tools to push the debris capture traps 410 slightly aside, to pass through.

FIG. 4C shows a view along a direction of fluid (for example blood) flow, demonstrating that debris flowing along the device will likely be trapped by a debris trap 410.

When the debris capture device 402 includes several open debris trap 410, covering the entire cross section of the stent 404, FIGS. 4A-4C demonstrates that debris larger than a mesh pore size of the debris traps 410 will be caught will be caught by the debris traps 410.

When the debris capture device 402 includes several open debris trap 410, even when the traps are pushed aside by medical tools, the debris mesh traps 410 cover the cross section area of the stent 404 not taken up by the medical tool, and debris larger than a mesh pore size of the debris traps 410 will be caught will be caught by the debris traps 410.

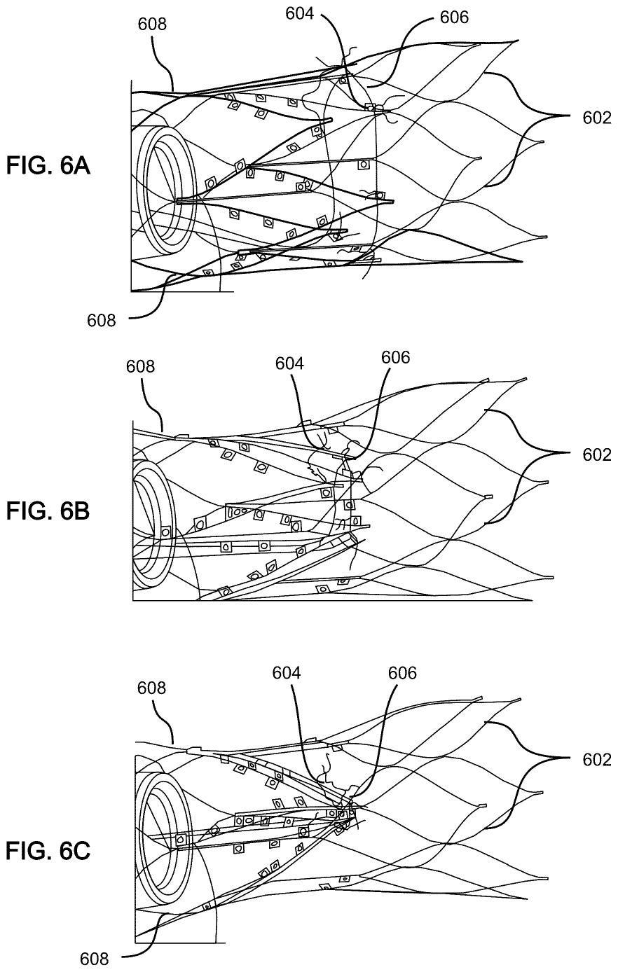

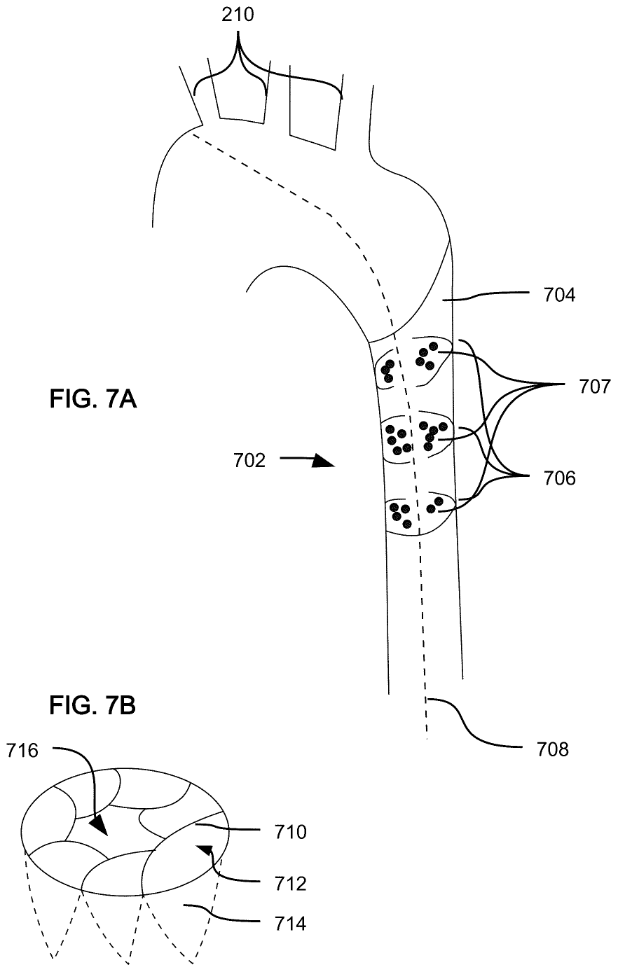

Reference is now made to FIG. 5A, which is a photo of a debris capture device according to an example embodiment.

FIG. 5A shows a debris capture device 500, including a stent 502 and leaves 504 or debris traps 504, or debris catching pockets 504.

FIG. 5A does not show a mesh which covers the leaves 504.

FIG. 5A shows an optional gradually-narrowing end 506 of the debris capture device 500. The gradually-narrowing end 506 potentially enables pulling the debris capture device 500 into a catheter for extraction from a patient's body. The gradually-narrowing end 506 potentially pulls the device 500 into the catheter and assists in compressing the device as it enters.

In some embodiments, the debris traps 504 are closed upon whatever debris the debris traps 504 trapped, when the device 500 is pulled into the catheter and extracted, together with the trapped debris, from the body.

Reference is now made to FIG. 5B, which is a simplified illustration of a debris capture device according to an example embodiment.

FIG. 5B shows a debris capture device 510, including a stent 512 and leaves 514 or debris traps 514, or debris catching pockets 514.

FIG. 5B does show a mesh which covers the leaves 514.

FIG. 5B shows an optional gradually-narrowing end 516 of the debris capture device 510. The gradually-narrowing end 516 potentially enables pulling the debris capture device 510 into a catheter for extraction from a patient's body. The gradually-narrowing end 516 potentially pulls the device 510 into the catheter and assists in compressing the device as it enters.

In some embodiments, the debris traps 514 are closed upon whatever debris the debris traps 514 trapped, when the device 510 is pulled into the catheter and extracted, together with the trapped debris, from the body.

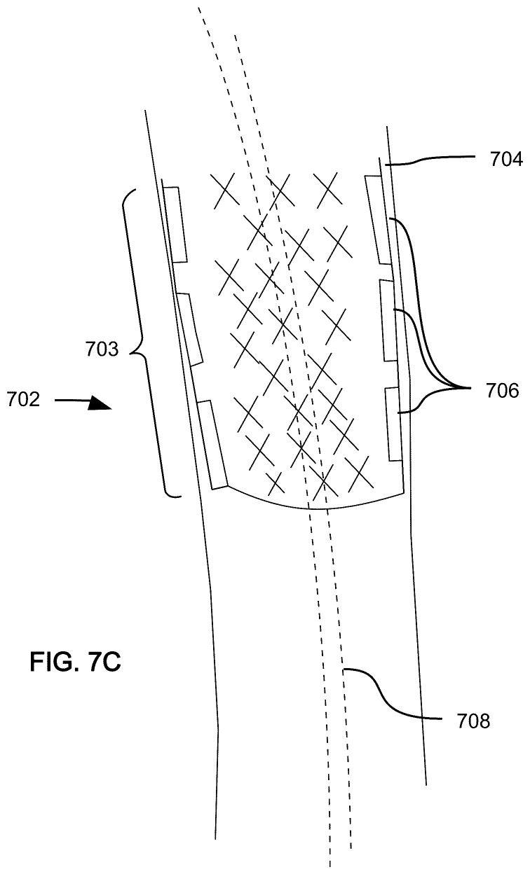

Reference is now made to FIGS. 6A-6C, which are photos of a debris capture device according to an example embodiment.

FIGS. 6A-6C show the debris capture device being gradually activated.

FIG. 6A-6C show a stent 602, tips 604 of debris traps or tips 604 of debris trapping leaves, a control wire guide 608, and a control wire 606 which is threaded through loops at the tips 604.

FIG. 6A shows the tips 604 of the leaves laying against the stent 602.

FIG. 6B shows the wire 606 pulling the tips 604 of the leaves closer to each other.

FIG. 6C shows the wire 606 pulling the tips 604 of the leaves to completely close a cross section of a lumen defined by the stent 602.

In some embodiments, the control wire guide 608 is optionally a lumen or pipe. In some embodiments, the control wire guide 608 is optionally constructed of nitinol or polymer.

In some embodiments, there are one or more control wire guides 608. The example embodiment shown in FIGS. 6A-6C shows two control wire guide 608.

In some embodiments, the number of control wire guide 608 is optionally an even number, for example 2, 4, 6, 8 and so on.

In some embodiments, the number of control wire guide 608 is not necessarily even.

When the cross section of the lumen defined by the stent 602 is completely closed, all fluid flowing through the lumen needs to pass through the leaves. In an embodiment where the leaves are covered by a mesh, all fluid flowing through the lumen needs to pass through the mesh.

It is noted that a medical tool can pass through the lumen of the device. It is clear that when the leaves lie against the stent 602, or the cross section is only partially closed, the medical tool can pass through the lumen of the device. It is pointed out that the wire 606 can be slightly released at any time, to allow the medical tool through, and the write can be slightly tightened to close upon the medical tool.

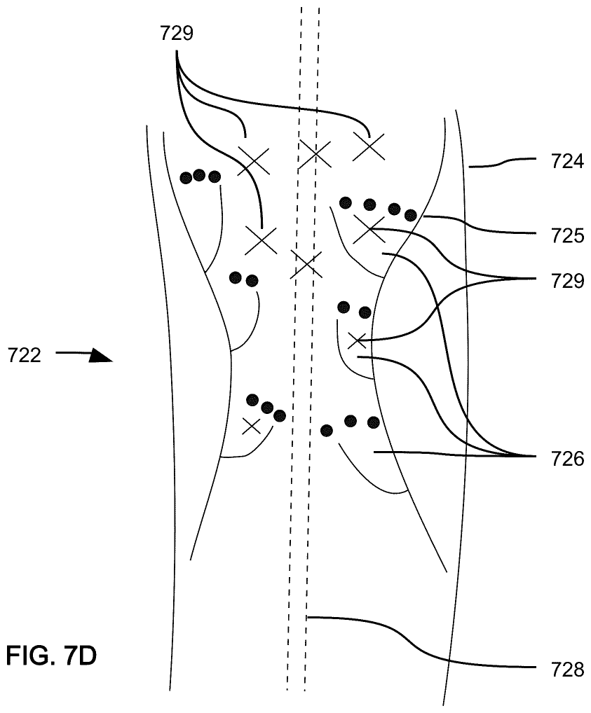

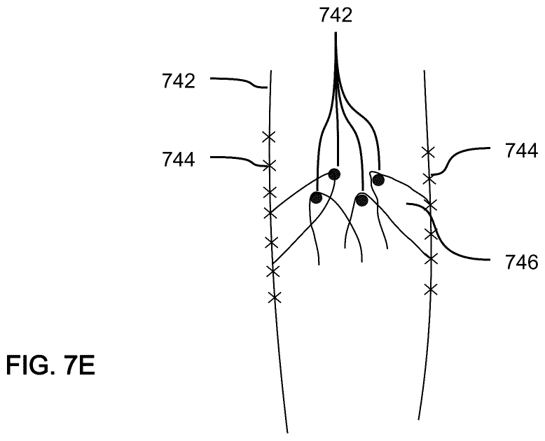

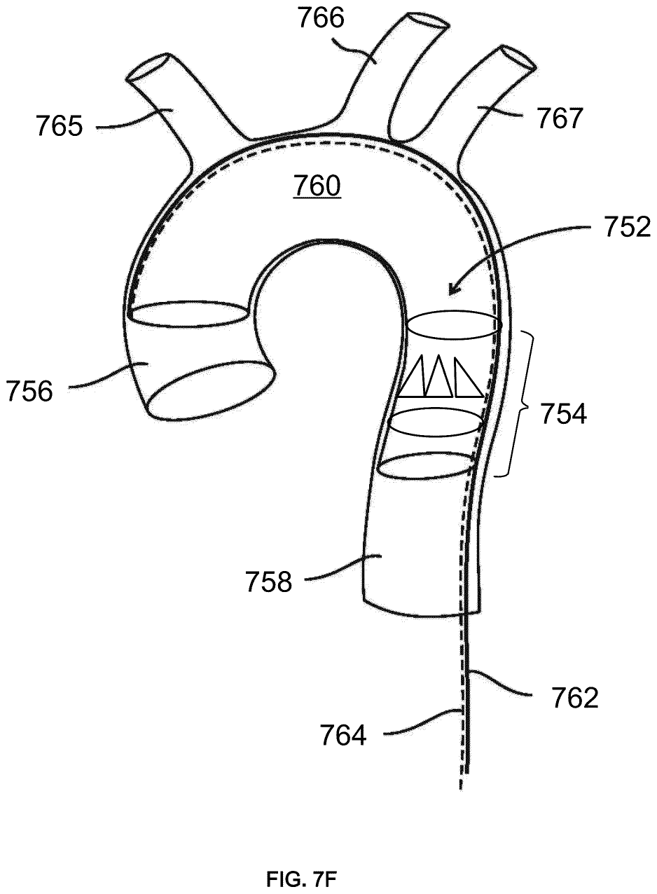

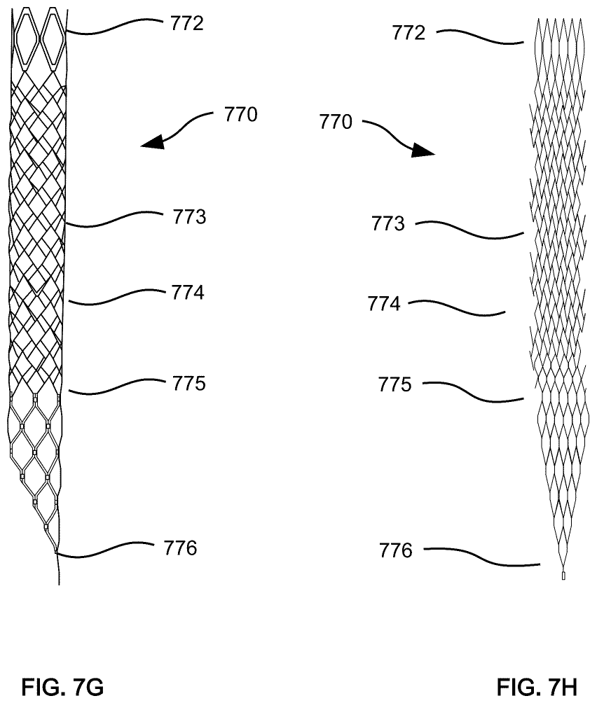

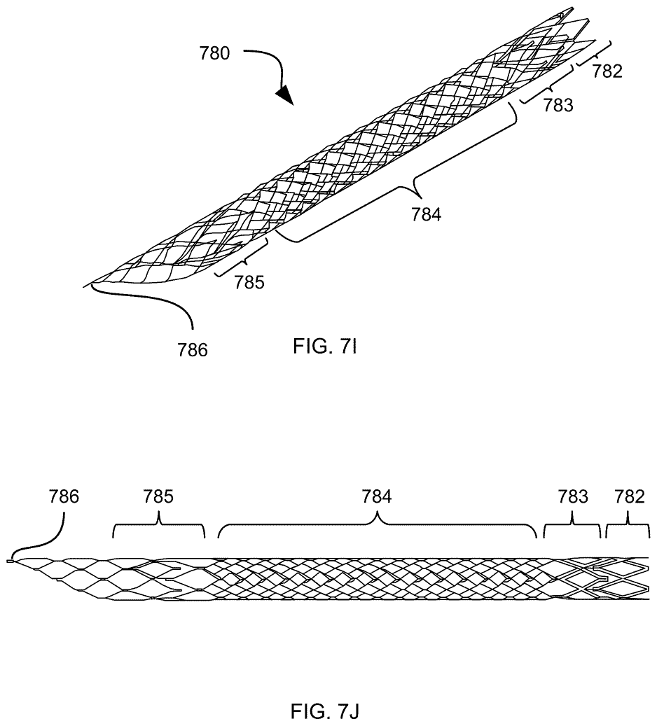

Reference is now made to FIGS. 7A-7C, which are simplified illustrations of a debris capture device located in an aorta according to an example embodiment.

FIG. 7A show a debris capture device 704, optionally having one or more levels of debris traps 706. The debris capture device 704 is optionally located in the descending aorta 702.

FIG. 7A also shows a medical tool 708, or a catheter 708, or a control wire 708 for a medical tool passing through the debris capture device 704, with the debris capture device 704 in an activated state, that is, the traps are open, and the leaves extend toward the middle of the descending aorta.

FIG. 7A shows debris particles 707 caught in the debris traps 706. When the debris traps 706 are optionally closed, the trapped debris 707 can optionally be extracted from the body together with the debris capture device 704.

FIG. 7B shows pockets 714 produced by an edge 710 of a trap being open, extending toward a middle 716 of a lumen defined by the debris capture device 704. Each trap presents an opening 712 which allows debris to flow down into the pocket 714.

FIG. 7C show the debris capture device 704, optionally having one or more levels of debris traps 706. The debris capture device 704 is optionally located in the descending aorta 702.

FIG. 7C also shows the medical tool 708, or the catheter 708, or the control wire 708 for a medical tool passing through the debris capture device 704, with the debris capture device 704 in a non-activated state, that is, the traps are closed or collapsed, and the leaves lie against walls of the debris capture device 704.

Reference is now made to FIG. 7D, which is a simplified illustration of a debris capture device located in an aorta according to an example embodiment.

FIG. 7D show a debris capture device 723, including a stent 724, mesh walls 725, optionally having one or more levels of debris traps 726. The debris capture device is optionally located in the descending aorta 722.

FIG. 7D also shows a medical tool 728, or a catheter 728, or a control wire 728 for a medical tool passing through the debris capture device, with the debris capture device in an activated state, that is, the traps 726 are open, and the leaves of the traps 726 extend toward the middle of the stent 724.

FIG. 7D also shows particles, marked by “X”s 729, flowing downstream along a lumen defined by the stent 724. Some of the particles 729 are shown inside the traps 726. When the debris traps 726 are optionally closed, the trapped debris 729 can optionally be extracted from the body together with the debris capture device 723.

In some embodiments, the mesh walls 725 of the debris capture device may be flexible enough that the mesh walls 725 bend inward, toward a center of the stent 724, when fluid flows through the debris capture device.

Reference is now made to FIG. 7E, which is a simplified illustration of a debris capture device located in an aorta according to an example embodiment.

FIG. 7E show a debris capture device, including a stent 744, optionally having one or more levels of debris traps 746. The debris capture device is optionally located in the descending aorta 742.

FIG. 7D also shows markers 742 attached to the debris traps 746. The markers 742 are selected to be opaque to one or more imaging modalities, so that a physician can view the whether the traps 746 are open or closed, using one of the imaging modalities.

Reference is now made to FIG. 7F, which is a simplified line drawing illustration of a device for aortic protection deployed in conjunction with a debris trapping device according to an example embodiment of the invention.

FIG. 7F shows an aortic protection device 752 such as, for example, described in above-mentioned International Patent Application Publication Number WO 2019/064223 of Brandeis, deployed in an aorta in conjunction with a debris trapping device 754, various embodiments of which are described herein.

It is noted that FIG. 7F shows the debris trapping device 754 deployed within a lumen defined by the aortic protection device 752. Various other locations of the debris trapping device 754 may be chosen, by way of some non-limiting examples:
not in the lumen defined by the aortic protection device 752, but upstream of the aortic protection device 752;
not in the lumen defined by the aortic protection device 752, but downstream of the aortic protection device 752;
partially in the lumen defined by the aortic protection device 752, and partially upstream of the aortic protection device 752; and
partially in the lumen defined by the aortic protection device 752, and partially downstream of the aortic protection device 752.

FIG. 7F shows a section of an aorta, the section extending from a heart-proximal side 756 to a heart-distal side 758, and includes the aortic arch.

FIG. 7F shows the aortic protection device 752 in the aorta. The aortic protection device 752 includes a mesh 760 in the aortic arch, and two optional wires 762 764 attached to the mesh 760. The mesh 760 covers artery exits to the brachiocephalic artery 765, the left common carotid artery 766 and the left subclavian artery 767.

The mesh 760 potentially blocks debris flowing with blood in the aorta from entering the above-mentioned arteries.

It is noted that in some embodiments the mesh 760 may optionally extend more or less than shown in FIG. 7F. By way of a non-limiting example the mesh 760 may extend much further on a heart-distal side of aorta, covering more artery exits from the aorta.

In some embodiments the mesh 760 is optionally controlled to change its porosity, for example from a larger pore size to a smaller pore size, providing better protection from debris entering the side arteries.

In some embodiments the mesh 760 is optionally controlled to change its porosity and to completely block blood from entering the side arteries.

In some embodiments the mesh 760 is optionally controlled to change its porosity for a limited amount of time, for example for a few seconds, for example 10 seconds, 30 seconds, 60 seconds, 90 seconds, up to a few minutes, for example two, three, four or five minutes.

In some embodiments the mesh 760 is optionally controlled to change its porosity and to completely block blood from entering the side arteries for a limited amount of time, for example for a few seconds, for example 10 seconds, 30 seconds, 60 seconds, 90 seconds, up to a few minutes, for example two, three, four or five minutes.

In some embodiments, the debris trapping device 754 is placed in the aorta after placing the aortic protection device 752 in the aorta. By way of a non-limiting example the aortic protection device 752 is optionally located and expanded first, and the debris trapping device 754 is optionally inserted into the lumen defined by the aortic protection device 752, located and deployed.

In some embodiments, by way of a non-limiting example when the debris trapping device 754 is located upstream or downstream of the aortic protection device 752, the order of insertion and/or location and/or deployment of the device may be independent of each other.

In some embodiments, the aortic protection device 752 and the debris trapping device 754 are optionally operated independently of each other.

In some embodiments, the aortic protection device 752 and/or the debris trapping device 754 are optionally operated in association with steps taken in medical procedures performed elsewhere in a patient's body, for example medical procedures performed on a cardiac valve, elsewhere on the heart, and even upstream of the left side of the heart or even upstream of the right side of the heart.

Reference is now made to FIGS. 7G and 7H, which are simplified line drawing illustrations of a device which includes a combination of aortic protection and debris trapping according to an example embodiment of the invention.

FIGS. 7G and 7H show a device 770, which includes an anchoring section 772, a stent-like lumen 773, a debris trap section 775, and an optional gradually-narrowing end 776 of the device 770.

FIG. 7G is an isometric view of the device 770, and FIG. 7H is a view of the device 770 laid out flat.

In some embodiments, the device 770 optionally includes a mesh 774 covering the lumen 773. The mesh 774 optionally acts to filter debris so as not to enter branching arteries, for example as described in above-mentioned International Patent Application Publication Number WO 2019/064223 of Brandeis.

Reference is now made to FIGS. 7I and 7J, which are simplified line drawing illustrations of a device which includes a combination of aortic protection and two locations or levels for debris trapping according to an example embodiment of the invention.

FIGS. 7I and 7J show a device 780, which includes an anchoring or fixation section 782, a first debris-trapping section 783, a stent-like lumen 784, a second debris-trapping section 785, and an optional gradually-narrowing end 786 of the device 780.

FIG. 7I is an isometric view of the device 780, and FIG. 7J is a side view of the device 780.

In some embodiments, the device 780 optionally includes a mesh (not shown) covering the stent-like lumen 784. The mesh optionally acts to filter debris so as not to enter branching arteries, for example as described in above-mentioned International Patent Application Publication Number WO 2019/064223 of Brandeis.

In some embodiments, the first debris-trapping section 783, and the second debris-trapping section 785 optionally include meshes for filtering fluid and trapping debris.

In some embodiments, the first debris-trapping section 783, and the second debris-trapping section 785 optionally include meshes with differently sized pores.

By way of a non-limiting example, in some embodiments, the pore size of the first debris-trapping section 783 may optionally be 30 microns.

In some embodiments, the first debris-trapping section 783 may optionally be activated separately from the activation of the second debris-trapping section 785.

In some embodiments, the first debris-trapping section 783 may optionally be activated when a medical procedure is known to produce a shower of debris, for example during a pace-up and/or a pace-down stage of a TAVI procedure.

By way of a non-limiting example, in some embodiments, the pore size of the second debris-trapping section 785 may optionally be 200 microns.

In some embodiments, the second debris-trapping section 785 may optionally be activated over a longer period, since the bigger-pored mesh produces a smaller pressure differential than, for example, 30 micron pores, and the body can withstand such a filter for a longer period.

In some embodiments, the stent-like lumen 784 is optionally covered by a mesh, optionally blocks debris flowing in a lumen from exiting sideways through the stent-like lumen 784.

By way of a non-limiting example, in some embodiments, the pore size of the optional mesh covering the stent-like lumen 784 may optionally be 120 microns.

By way of a non-limiting example, in some embodiments, the pore size of the optional mesh covering the stent-like lumen 784 may optionally be a size between a size of the pores of the first debris-trapping section 783 and the second debris-trapping section 785.

FIGS. 7I and 7J do not show optional control wires which may be used to control one or more of:
activating and deactivation the anchoring or fixation action of the anchoring or fixation section 782;
activating and deactivation the debris trapping of the first debris-trapping section 783;
changing mesh size of an optional mesh covering the stent-like lumen 784; activating and deactivation the debris trapping of the second debris-trapping section 785; and
pushing or pulling the optional gradually-narrowing end 786 of the device 780, to locate the device in place and/or extract the device.

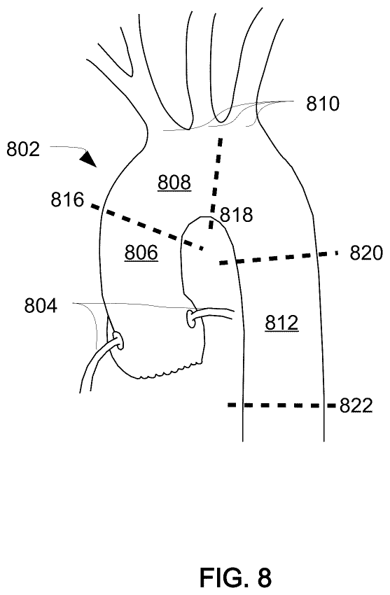

Reference is now made to FIG. 8 which is a simplified illustration of potential locations for a debris trapping device in an aorta according to some example embodiments.

FIG. 8 shows an aorta 802, and further details potions of the aorta: the ascending aorta 806, the aortic arch 808 and the descending aorta 812, including the thoracic aorta and the abdominal aorta.

FIG. 8 also shows locations of openings to the coronary arteries 804, and to the arteries leading to the brain-the brachiocephalic artery, the left common carotid artery, and the left subclavian artery.

FIG. 8 shows various potential locations which may be chosen for placing the debris trapping device-a first location 816 in the ascending aorta 806, a second location 818 in the aortic arch 808, a third location 820 in the descending aorta 812, and a fourth location 822 further downstream, in the thoracic aorta or the abdominal aorta.

In some embodiments, a debris trapping device is placed in a vein, to prevent debris from reaching the lungs.

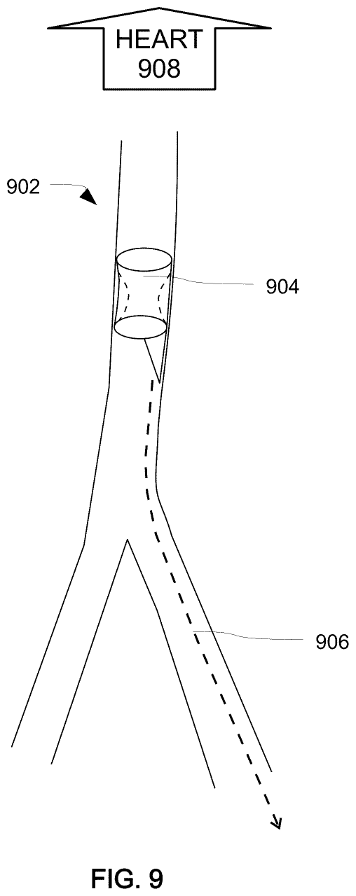

Reference is now made to FIG. 9 which is a simplified illustration of a potential location for a debris trapping device in a vein according to some example embodiments.

FIG. 9 shows the Vena Cava 902, and a debris trapping device 904 located in the Vena Cava 902. The Vena Cava 902 collects blood from veins to a right atrium of the heart 908. The heart pumps the blood to the lungs. If debris reaches the lungs it may be very detrimental.

In some embodiments, the debris trapping device 904 is placed in the Vena Cava 902, to block such debris.

In some embodiments, the debris trapping device 904 traps debris and is eventually withdrawn from a body, together with the debris.

In some embodiments, the debris traps in the debris trapping device 904 are closed to contain trapped debris prior to withdrawing from the body together with the debris.

In some embodiments, an anti-coagulant is optionally administered, to break up and/or dissolve blood clots trapped by the device before extracting the device from the body.

Following are descriptions of some non-limiting example methods of using a debris capture device.

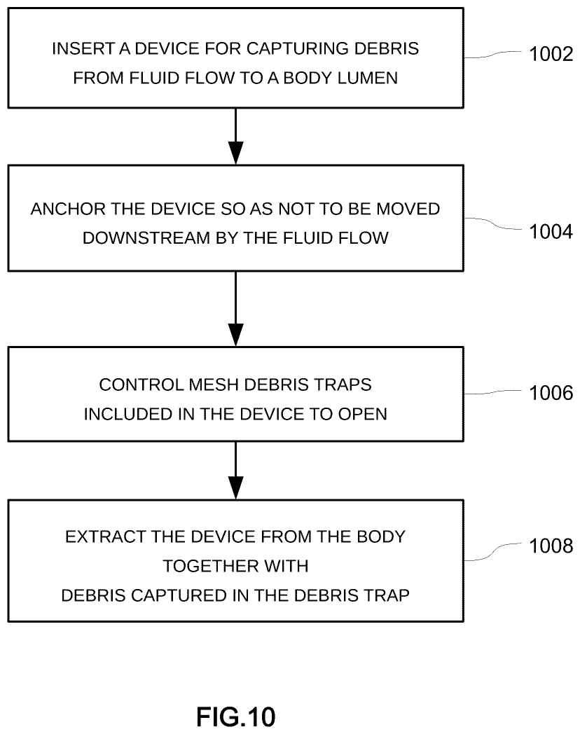

Reference is now made to FIG. 10, which is a simplified flow chart illustration of a method for collecting debris according to an example embodiment.

FIG. 10 includes:
inserting a device for capturing debris from fluid flow to a body lumen (1002),
anchoring the device so as not to be moved downstream by the fluid flow (1004),
controlling mesh debris traps included in the device to open (1006), and
extracting the device from the body together with debris captured in the debris trap (1008).

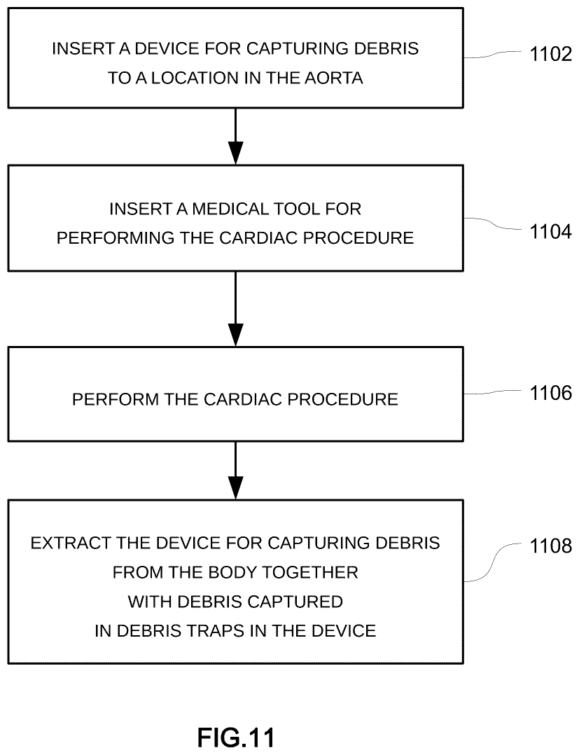

Reference is now made to FIG. 11, which is a simplified flow chart illustration of a method for collecting debris during a cardiac procedure according to an example embodiment.
inserting a device for capturing debris to a location in the aorta (1102),
inserting a medical tool for performing the cardiac procedure (1104),
performing the cardiac procedure (1106) and
extracting the device for capturing debris from the body together with debris captured in debris traps in the device (1108).

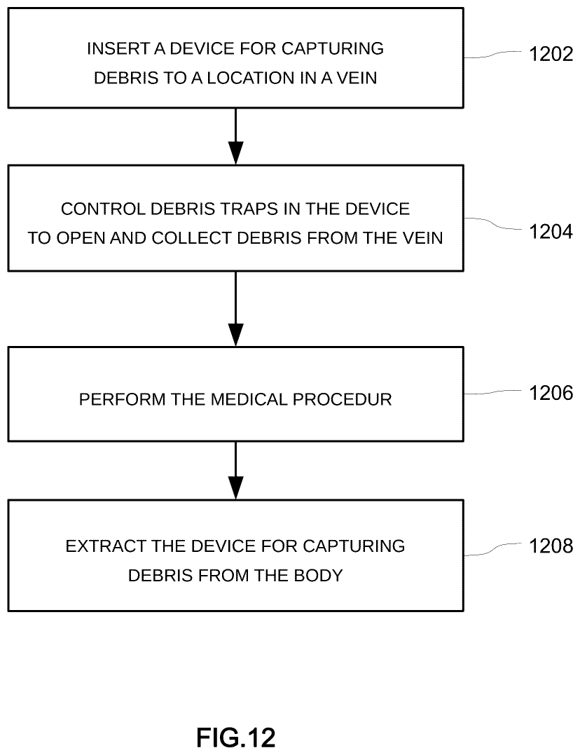

Reference is now made to FIG. 12, which is a simplified flow chart illustration of a method for preventing debris from reaching a lung according to an example embodiment.
inserting a device for capturing debris to a location in a vein (1202),
controlling debris traps in the device to open and collect debris from the vein (1204),
performing the medical procedure (1206) and
extracting the device for capturing debris from the body (1208).

It is expected that during the life of a patent maturing from this application many relevant types of mesh will be developed and the scope of the term mesh is intended to include all such new technologies a priori.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device for capturing debris from blood flow in an aorta, the device comprising:
an expandable stent shaped and sized to expand against walls of a body lumen;
a plurality of mesh debris traps attached to the stent;
a control wire attached to one or more of the mesh debris traps, configured to control opening of the mesh debris traps;
wherein the mesh debris traps are arranged to be normally closed even when the device is expanded against walls of the body lumen; and
wherein the mesh debris traps are arranged to be open by actuating said control wire.

2. The device according to claim 1 wherein the expandable stent is shaped and sized to expand against walls of an aorta and/or expand against walls of a Vena Cava.

3. (canceled)

4. The device according to claim 1, wherein the control wire is sized to extend from a location of the device in a patient's body to outside the patient's body.

5. The device according to claim 1, wherein the mesh debris traps are arranged to be controlled by the control wire from outside a patient's body.

6. The device according to claim 1, wherein at least some of the mesh debris traps are arranged to be controlled separately from at least some other mesh debris traps.

7. The device according to claim 1, comprising a plurality of control wires, each control wire configured to control a separate group of the mesh debris traps.

8. (canceled)

9. The device according to claim 1, wherein the mesh debris traps are configured for one or more of:
a. to allow passage of medical tools through a tubular lumen defined by an inside of the device when the device is expanded against walls of the body lumen;
b. such that mesh debris trap openings are distributed at different distances along a direction of blood flow through the device.

10. The device according to claim 1, wherein the mesh debris traps comprise one or more of:
a. mesh leaves attached at their base to stent walls, and edges positioned upstream of the base of the mesh leaves;
b. mesh leaves attached at their base to stent walls, and edges positioned downstream of the base of the mesh leaves;
c. markers to enable detecting whether the mesh debris traps are open or closed;
d. a mesh with pore sizes in a range between 1000 and 30 microns;
e. a loop for threading a control wire therethrough, the control wire serving to open the mesh debris trap.

11-15. (canceled)

16. The device according to claim 1, wherein mesh debris traps are arranged in one or more ways as following:
a. longitudinally along the device such that, when open, openings of open mesh debris traps overlap, as viewed along a direction of blood flow;
b. such that larger pore sizes are upstream of smaller pore sizes;
c. distributed at different distances along a direction of blood flow comprise different sizes of mesh pore openings.

17. (canceled)

18. The device according to claim 1, wherein mesh debris trap openings are one or more of:
a. at same distances along a direction of blood flow comprise different sizes of mesh pore openings;
b. flexible, the mesh debris trap openings allowing surgical tools to bend them and pass along the device.

19-20. (canceled)

21. The device according to claim 1, wherein expansion of the stent against walls of the body lumen anchors the device to resist movement along a direction of blood flow.

22. The device according to claim 1, wherein the device is anchored to resist movement along a direction of blood flow by connection to an anchor stent expanded against lumen walls upstream of the device;
wherein the anchor stent is shaped and sized for anchoring upstream of a brachiocephalic trunk;
wherein the anchor stent is shaped and sized for anchoring upstream of the carotid arteries.

23-24. (canceled)

25. The device according to claim 1, wherein the device is configured to one or more of:
a. be attached to an aortic protection device;
b. be as a part of an aortic protection device.

26. (canceled)

27. A method for collecting debris, the method comprising:
inserting a device for capturing debris from fluid flow to a body lumen;
anchoring the device so as not to be moved downstream by the fluid flow;
controlling mesh debris traps included in the device to open; and
extracting the device from the body together with debris captured in the debris trap

28. The method according to claim 27, wherein the body lumen is at least one of a blood vessel, an artery and an aorta.

29-30. (canceled)

31. The method according to claim 28, and further comprising inserting tools for performing a cardiac procedure and/or a surgical operation along the device and upstream of the device.

32. The method according to claim 28, and further comprising performing the cardiac procedure following the opening of the mesh debris traps.

33. The method according to claim 27, wherein the device is used in addition to use of an aortic protection device.

34. (canceled)

35. The method according to claim 27, wherein the method is performed upon a patient prior to performing a medical procedure expected to release debris or produce debris into veins.

36. The method according to claim 27, and further comprising closing the mesh debris traps prior to extracting the device from the body.

37. (canceled)

38. The method according to claim 27, wherein the opening the mesh debris traps comprises controlling just a sub-group of the mesh debris traps.

39-50. (canceled)
